(19) United States
(12) Patent Application Publication
(10) Pub. No.: US 2006/0065010 A1
(43) Pub. Date: Mar. 30, 2006

(54) Cover for cold foods

(76) Inventor: Lawrence Mackiewicz, Kingston, NY (US)

Correspondence Address: Donald R. Heiner, Esq., 71 Mount Vernon Street, Ridgefield Park, NJ, 07660, US

(21) Appl. No.: 10/952,998

(22) Filed: Sep. 30, 2004

(51) Int. Cl. F25D 3/08; A61F 7/00

(52) U.S. Cl. 62/457.2; 62/371

(57) ABSTRACT

A cover for the protection and displaying cooked, non-cooked, cooled and non-cooled foods wherein the cover is of a clear or slightly tinted material such that the food can be observed by a person wishing to serve themselves. The cover has a handle for rotating the cover ninety (90) degrees to a substantially vertical position wherein it rests on another handle or the like. Disposed within this cover is a gel pack which operates as a cooling device. Spacers are provided internally on the cover near the inside top thereof to prevent the gel pack from touching the top of the cover and triangular shaped protrusions are associated with the cover to prevent the gel pack from unintentionally sliding out of it if a person picks up the cover by the back handle. The gel pack is kept in place by a support bar, which may swivel, and which acts as support for the gel pack when the cover is in its lowered or cooling position and which support bar may swivel up and out of the way when in the raised or non-cooling position. An air space exists between the top of the gel pack and the inside of the cover to allow for a convection current to become established within the display cover. The shape of the cover is designed for the convection current to flow easily.

### BACKGROUND OF THE INVENTION

1. Field of the Invention

The present invention generally involves the field of technology pertaining to a cover essentially for cool or cold foods wherein the cover is either clear or slightly tinted such that the food covered by the cover can be seen by a person wishing to serve themselves without first removing the cover.

The cover has a front and back handle for rotating it 90 degrees from a substantially horizontal position covering the food to a substantially vertical position wherein it rests on another handle or some other device. This allows a person carrying a plate or the like to rotate the cover, take a desired quantity of food and then rotate the cover back into its horizontal position covering the food without the necessity of putting down the plate or tray or the like.

Disposed within the cover is a gel pack or the like which operates as a cooling medium for the food to be preserved.

This gel pack or cooling device is disposed internally of the cover and spacers are provided to prevent the gel pack from touching the top of the cover and, in addition, triangular shaped protrusions are also associated with the top inside of the cover to prevent the gel pack from unintentionally sliding out of it if a person picks up the cover by the back handle rather than the front handle.

One of the principle elements of the invention is a support bar disposed in the rear upper portion of the cover. This support bar may swivel and also acts as support for the gel pack when the cover is in its lowered or cooling or horizontal position. The support bar may swivel up and out of the way when in the raised or non-cooling position.

By virtue of the space between the top of the cover and the top of gel pack an air space exists inside the cover to allow for a convection current to become established within the display cover.

2. Description of the Prior Art

A search of the prior art uncovered patents to Conklin, U.S. Pat. No. 4,019,340; Branscum, U.S. Pat. No. 4,024,731; Buss, U.S. Pat. No. 4,213,310; McClure, U.S. Pat. No. 5,307,647; Heverly; U.S. Pat. No. 5,701,757; Roth, et al, U.S. Pat. No. 6,601,403.

None of the references cited above disclose a “see through” cover nor the concept of being able to rotate a food cover to a vertical position while it stands on its own leg or handle. No mention was found of a “convection current”.

Of some interest is the patent to Conklin, U.S. Pat. No. 4,019,340 wherein “the cover includes a frame having inner and outer ends and the passage extending through the frame”. Please note column 4, lines 3-8 which described a packaged refrigerant gel. However, in this patent, cover 15 itself is actually placed into a freezer to freeze the refrigerant gel 47. In column 4, beginning at line 31, it is noted that “if the upper wall 27 is removable from the frame 23, the refrigerant gel 47 and its container 45 can be removed from the cover and frozen”.

In this case, there is no tilt back lid or clear plastic such as described in the instant invention. Further, it does not disclose a bracket for holding a gel pack.

The Branscum patent U.S. Pat. No. 4,024,731 describes an insulated container with freezable lid-mounted bottles 22 as shown specifically in FIG. 1 and FIG. 2.

The Buss patent U.S. Pat. No. 4,213,310 discloses a thermal container with a quick-release lid-mounted flask 12 as shown in FIGS. 2-4 for thermally heating or cooling the contents of cavity 56 as shown in column 2, lines 48-51. “A thermal flask 12 is positioned in chamber 54 of lid 14 for thermally heating or cooling the contents in the cavity 56.”

The McClure patent U.S. Pat. No. 5,307,647 relates to a food serving refrigerant device with refrigerant units 36 in its base. The temperature maintaining refrigerant units are disposed within a refrigerant cavity configured in a base of the device in interfitted puzzle-like fashion.

The Heverly patent U.S. Pat. No. 5,701,757 is a portable refrigerated food container with a gel pack 30 in its base. More specifically, “a gel pack 30 removably positioned on the interior bottom surface which is freezable and an inner pan 40 formed to a shape similar to outer pan 20 which retains food 12 including at least one handle 46 secured to the upper exterior portion, and slidably positionable within the outer pan 20 juxtaposed to the gel pack 30.

The Roth, et al patent U.S. Pat. No. 6,601,403 relates to a thermal storage lid as best seen in FIG. 1. Note the compartment 34 for gel pack 40 and column 2, lines 40-56 basically describe the invention.

### SUMMARY OF THE INVENTION

According to the present invention there is provided a cover, preferably of clear or slightly tinted material, for covering cool or cold foods, and wherein the cover is rotatable 90 degrees from its position covering such cold foods. The invention may be used to preserve both cooled and non-cooled foods at a given temperature and whereby a convection current is automatically provided within the cover itself.

The cover can be tipped from its normal, horizontal position covering the food to a 90 degree serving position or anywhere in between.

Such a clear or slightly tinted display cover allows for visibility of the food being presented or served. While other cooling mediums may be used a gel pack is the preferred embodiment.

The cover of the invention comprises a front handle which allows the cover to be easily tipped from a horizontal position to a vertical serving position or any position in between.

In addition to the front handle there is a rear handle or other device which may act as a stand when the cover is placed in a vertical position.

Molded into or otherwise disposed within the cover and near the top thereof are a plurality of spacers of any convenient configuration and usually made from the same material as the cover itself. This would not preclude the use of other materials. The gel pack sits on these spacers and prevents the gel pack from contacting the upper inside of the cover. Further, these same spacers will prevent the gel pack from contacting the inner vertical surface of the top of the cover. Thus, an airspace is provided between the rear and top of the gel pack and the top and rear wall of the cover thereby allowing a channel for the flow of cold air or a convection current.

Further, in addition to the foregoing mentioned spacers triangular shaped protrusions or stops are provided at the inner rear top of the cover which are triangular shaped protrusions to prevent the gel pack from unintentionally sliding out if a person raises the cover by the back rather than the front handle. All of theses stops and spacers collectively act as a cradle or positioner for the gel pack. It should be obvious that the protrusions or stops may be of any convenient shape and not necessarily limited to a triangular shape.

Further, and most importantly, a swivel support bar is provided near the upper, rearward, back of the cover which, acts or cooperates with the stop elements for positioning the gel pack in the cover. This support bar may be fastened by any well known means within the cover.

It should be obvious that this cover may use any type of handles and not just those shown in the drawings. Further, something other than a handle, such as two separate legs, may be used to support the cover in its vertical position. Still further, the support bar may be made of any suitable material. The bar may also be removable rather than merely swiveling from the non-cooled function.

As follows it is therefore an object of the present invention to provide a cover for displayed foods.

It is another object of the invention to provide such a food cover used especially for cool or cold foods to be served.

It is yet a further object of the invention to provide such a food cover wherein the cover is rotatable from a horizontal position covering the food to a vertical position uncovering the food and allowing for a person to serve themselves without the necessity of further moving the cover or putting down their tray or plate.

It is still a further object of the invention to provide such a food cover that is made of clear or slightly tinted materials such that the food displayed can be viewed without rotating the cover.

It is still a further object of the invention to provide such a cover further having a cooling device such as gel pack disposed therein for keeping the displayed food cool.

It is still a further object of the invention to provide such a cover whereby the gel pack can be easily removed and replaced.

These and other objects, features and advantages of the invention shall become apparent from the following detailed description of a preferred embodiment thereof when taken in conjunction with the drawings, wherein like referenced characters refer to corresponding parts in the several views.

### BRIEF DESCRIPTION OF THE DRAWINGS

FIG. 1 is a perspective view of the cover of the instant invention.

FIG. 2 is a front view of the cover of the instant invention.

FIG. 3 is a side view of the instant invention.

FIG. 4 is a view similar to FIG. 2 but showing a rearview of the present invention.

FIG. 5 is a front cross-section of the instant invention showing the details of the cradle for the gel pack or other cooling medium.

FIG. 6 is a side cross-section of the instant invention similar to FIG. 3.

FIG. 7 is a front cross-sectional view, similar to FIG. 2, disclosing the details of the specifics of the invention.

FIG. 8 is a side cross-sectional detail of the present invention.

FIG. 9 discloses the cover in both the horizontal and vertical position and sitting on a table.

### DETAILED DESCRIPTION OF THE PREFERRED EMBODIMENT

A clear or slightly tinted cover preferably of a thermal plastic material, or any other suitable material that will serve this purpose, and whose main purpose is to cover and keep cool cold foods displayed on a table or stand or any other well known server will now be described with reference to the drawings.

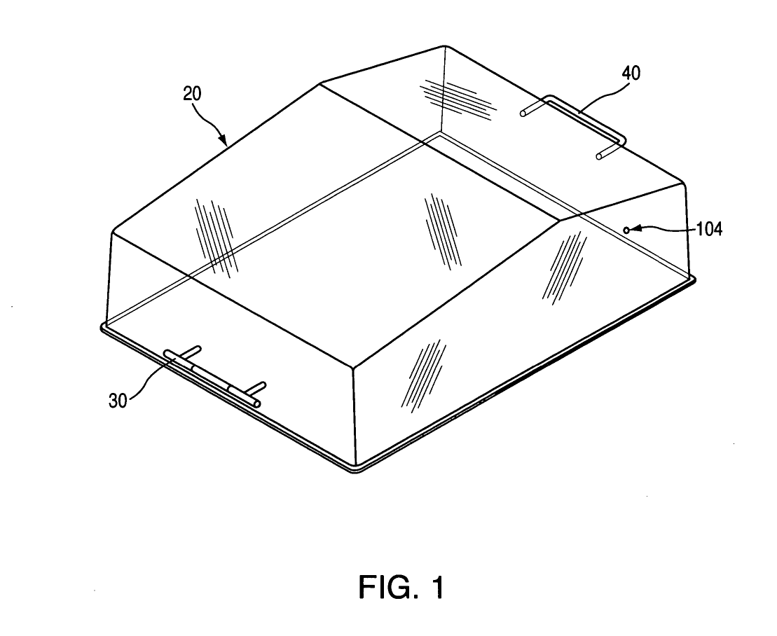

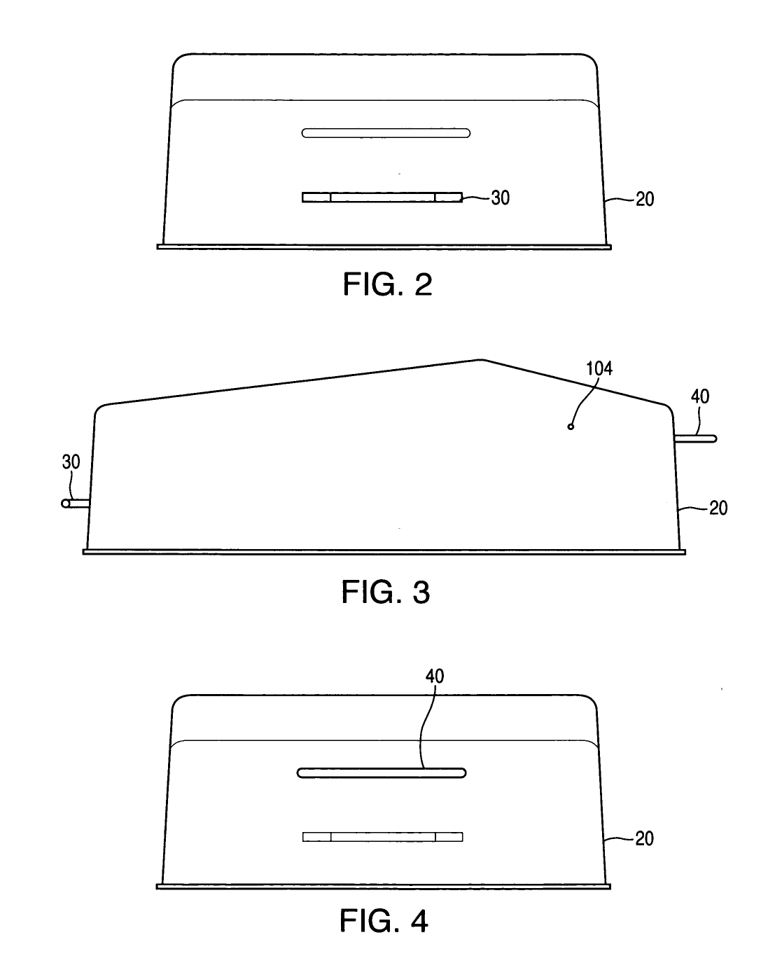

The cover of the instant invention is shown in all of the Figures as number 20 and having the basic configuration as shown in all of these Figures and especially as shown in FIGS. 1, 3 and 6. This cover 20, as indicated above, comprises a clear or slightly tinted material, basically for covering foods to be kept cold but which obviously can be used any foods to be covered. The purpose of using a clear or slightly tinted thermal plastic or other material allows a person or viewer or someone wishing to serve themselves some food to view the food without the necessity of removing the cover.

A flange or lip 50 surrounds the entire lower edge of cover 20.

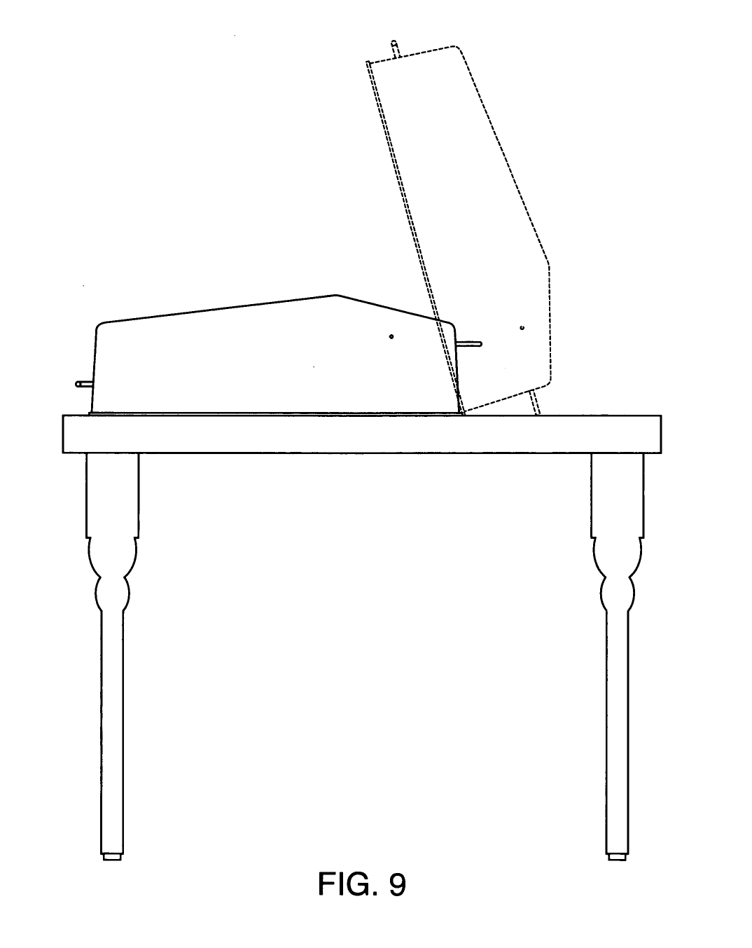

Attached to the front of cover 20 is a handle 30 for grasping and rotating the cover 20 into the vertical position in FIG. 9. In addition to its use to rotate the cover to a vertical position this handle 30 may also serve as a carrying handle.

A support leg 40 is disposed on the rear panel of cover 20 functions to serve as a support for cover 20 when it is rotated into its vertical position while someone is removing the cool food from the display.

Any other suitable means such as two (2) independent support legs may be employed to provide the necessary support for cover 20 when it is tipped into its vertical or serving position.

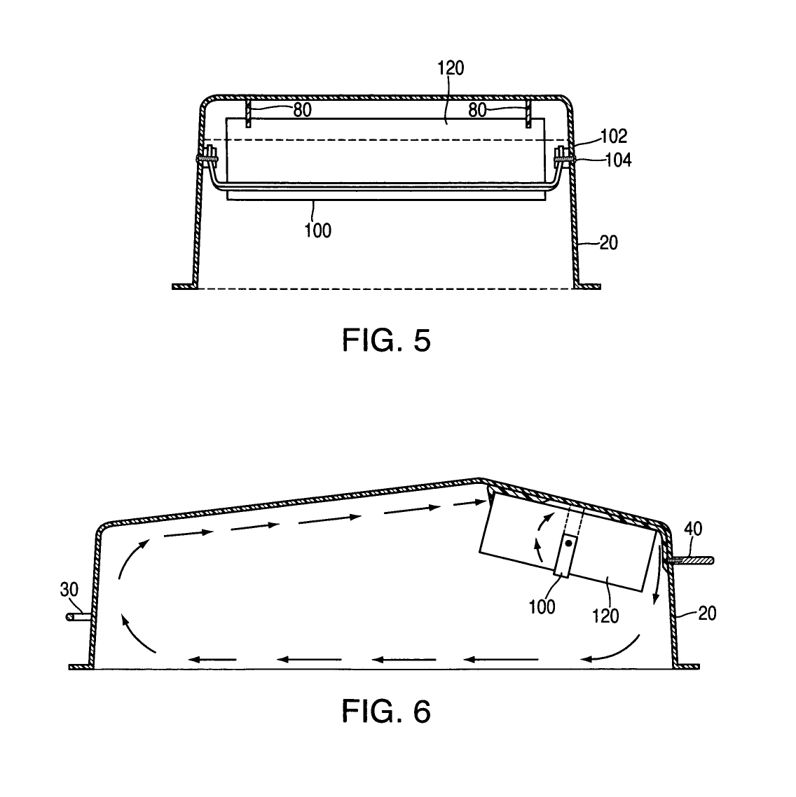

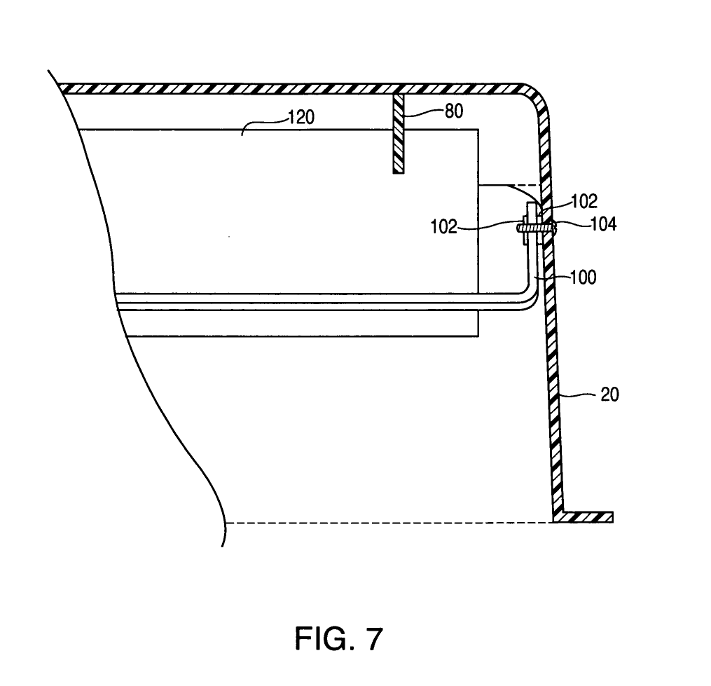

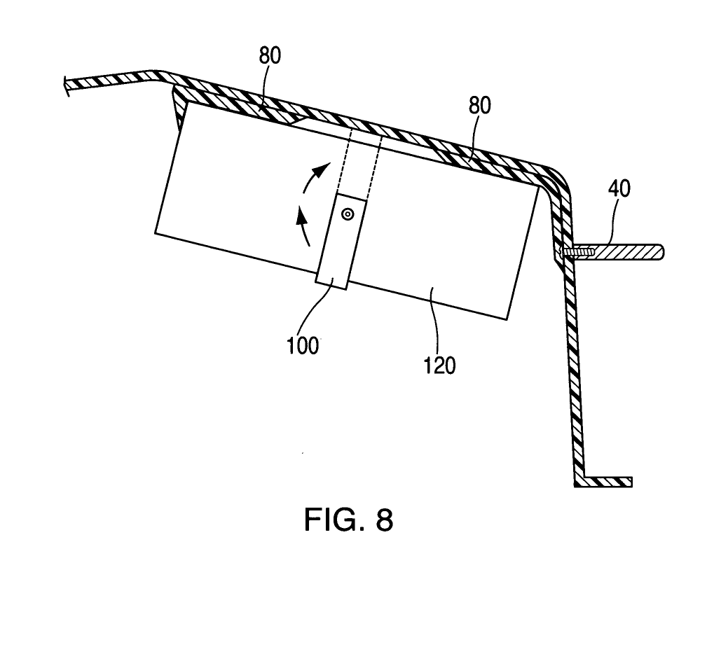

For keeping the food cool a cooling medium such as gel pack 120 shown in FIGS. 5-8 is disposed internally of cover 20 and is held in place by means of a combination of a rotatable support bar 100 as best seen in FIGS. 6, 7, and 8. While any well known means may be employed to hold the support bar in place there is shown in the drawings washers 102 and rivet 104. This support bar helps support the gel pack when the cover is used in the cool mode and it can rotate up when in a non-cool mode as best seen in FIGS. 6 and 8.

In addition to the rotatable support bar 100 the gel pack is also held in place by means of spacers 80 which also serve to prevent the gel pack from touching the top of the cover and also by means of triangular shaped protrusions associated with the top inside of the cover. to prevent the gel pack from unintentionally sliding out of it if a person picks up the cover by the back handle 40 rather than the front handle 30.

One of the principal elements of the invention is the support bar disposed in the rear upper portion of the cover. As previously discussed, the support bar may swivel and also acts as support for the gel pack when the cover is in its lowered or cooling or horizontal position. As previously discussed, this support bar may swivel up and out of the way when the cover is in the raised or non-cooling position.

The natural space that exists between the top of cover 20 and the top of gel pack 120 an airspace exists inside the cover, near the top thereof, to allow for a convection current to become established within the display cover. The shape of the cover is designed for the convection current to flow easily. This aids in the cooling process.

The cooling medium or gel pack is cradled or positioned into the combination of the plurality of stops molded into or otherwise disposed within the cover and near the top thereof, the spacers provided at the inner rear top of the cover which are preferably, but not necessarily of triangular shape, and the swivel support bar.

### Operation of the Invention

In operation, the cover is placed over food, preferably food to be kept cool, and in a horizontal position. As discussed, this cover can be tipped approximately 90 degrees into an upright serving position. A person, wishing to serve themselves, and usually having a plate in one hand, grabs the front handle and tips the cover into an upright position thereby exposing the food. After serving themselves the food the user then rotates the cover downwardly approximately 90 degrees thereby again covering the food. As explained, the cover can be a “see through” of clear or slightly tinted plastic or other suitable material.

The food is kept cool by means of the gel pack or other cooling instrument that is disposed in the cradle as discussed above inside the cover, near the top, and near the rear of the cover.

The cooling element or gel pack can be removed at any given time as discussed above and replaced with a new gel pack.

Though the invention has been described and illustrated with reference to a preferred embodiment thereof, those skilled in the art will appreciate that various changes and modifications in shape, size, composition and arrangement of parts, may be resorted to without departing from the spirit of the invention or scope of the subjoined claims.

What is claimed:

1. A cover for a food display, especially cold foods, comprising:
(a) a front handle for rotating said cover from a normal horizontal position covering the food to a vertical serving position;
(b) a rear handle which acts as a stand for said cover when said cover is in a vertical or 90 degree serving position;
(c) a cooling medium disposed in said cover for keeping said food cool;
(d) a plurality of spacers disposed on the top inside of said cover to prevent said cooling medium from contacting the upper inside of said cover;
(e) a plurality of stops disposed on the inner rear top of said cover to prevent said cooling medium from unintentionally sliding out if said cover is raised by said back rather than said front handle;
(f) an airspace disposed between the rear and top of said cooling medium and the rear wall of said cover thereby allowing a channel for the flow of cold air or a convection current within said cover when in its horizontal food covering position; and
(g) a swivel support bar disposed within said cover near the upper, rearward, back of said cover which support bar cooperating with said stops and said spacers positions said cooling medium in said cover.

2. The cover of claim 1 wherein said cover is either clear or slightly tinted such that said food display may be viewed by a person wishing to serve themselves without first removing said cover from said food display.

3. The cover of claim 2 wherein said cooling medium is a gel pack configured to be held in place by said swivel support bar, said stops and said spacers.

4. The cover of claim 3 wherein said swivel support bar may swivel up and out of the way when said cover is in a raised or non-cooling position.

5. The cover of claim 3 wherein said support bar may be removable from said cover.
